(19) United States
(12) Patent Application Publication
(10) Pub. No.: US 2013/0337047 A1
(43) Pub. Date: Dec. 19, 2013

(54) COMPOSITIONS FOR TREATING ORAL AND PERIODONTAL INFECTIONS

(71) Applicant: ZarzaTech, Inc., Cambridge, MA (US)

(72) Inventor: Sonia DIVNEY, Boston, MA (US)

(73) Assignee: ZARZATECH, INC., Cambridge, MA (US)

(21) Appl. No.: 13/918,441

(22) Filed: Jun. 14, 2013

Related U.S. Application Data
(60) Provisional application No. 61/660,225, filed on Jun. 15, 2012.

(51) Int. Cl. A61K 9/127; A61K 31/4164

(52) U.S. Cl. CPC A61K 9/127; A61K 31/4164
USPC 424/450; 514/400

(57) ABSTRACT

The present invention provides novel compositions, and their methods of manufacture and use, comprising a formulation with a mucoadhesive polymer carrying a positive charge such as low molecular weight chitosan, a negatively charged lipid and H2 antagonist useful in the prevention and treatment of oral infectious disease in a nanotechnology or coated liposome carrier.

### CROSS REFERENCE TO RELATED APPLICATIONS

This application claims the benefit of U.S. Provisional Application No. 61/660,225, filed on Jun. 15, 2012, “Formulations and Methods for Novel Oral Treatment”, the contents of which are hereby incorporated by reference herein.

### FIELD OF THE INVENTION

The present invention provides novel compositions, and their methods of manufacture and use, comprising a formulation with a mucoadhesive polymer carrying a positive charge such as low molecular weight chitosan, a negatively charged lipid and H2 antagonist useful in the prevention and treatment of oral infectious disease in a nanotechnology or coated liposome carrier.

### BACKGROUND OF THE INVENTION

The contents of all references cited in this application are herein incorporated by reference.

Periodontal disease is a group of diseases affecting the periodontium, the tissues surrounding and supporting the teeth. Any inherited or acquired disorder of the periodontium can be defined as a periodontal disease, but the term usually refers to the common inflammatory disorders of gingivitis and periodontitis that are caused by pathogenic microflora in the biofilm or dental plaque that forms adjacent to the teeth on a daily basis. The inflammation associated with periodontal disease is caused by the host response to specific microorganisms Tissue and gingival crevicular fluid levels of histamine are reported to be increased in patients with gingivitis and periodontitis. Histamine alters a variety of neutrophil, macrophage, and monocyte functions mediated through the binding of H2 receptors on the cell surface. Schenkein, H., The Pathogenesis of Periodontal Diseases, Academy Reports, Journal of Periodontology, 457-466 (1999).

Because periodontitis is a chronic inflammatory disease that can be caused by injury or bacteria, it follows that two mechanisms of treating periodontal disease would be to control either the bacterial infection or to control the inflammatory or so-called host-factor related aspect of the disease.

The mechanical removal of the microbial biofilm has been the standard for prevention and treatment of periodontal conditions since ancient times. Although the microbial plaque is necessary for gingivitis and periodontitis to occur, it is not sufficient on its own to cause periodontitis. Differences in host response to the microbial biofilm appear to be the critical determinant of disease severity, extent and progression. The difference in host response is the reason why, in the absence of plaque control, a patient will develop mild versus severe gingivitis, and further, whether or not the presence of a chronic plaque burden causes gingivitis to progress to periodontitis. Reinforcement of the leukocyte barrier against oral pathogens is critical to preventing both the transition from gingivitis to periodontitis and periodontal disease progression. Attstrom, R., The Roles of Gingival Epithelium and Phagocytosing Leukocytes in Gingival Defence, Journal of Clinical Periodontology, 2:25-32 (1975).

Cimetidine is a specific histamine H2 receptor antagonist, which inhibits the histamine stimulated release of gastric acid and is therefore used widely for the treatment of peptic ulcer. Kenyon, G. S., et al., Cimetidine and the Gastric Mucosal Barrier, Gut, 18:631-635 (1977). Cimetidine eliminates the downstream inhibitory actions of histamine on chemotaxis, phagocytosis, superoxide anion production and the production of TNF-α and IL-12 by macrophages.

Earlier in vivo studies indicated that cimetidine reduced gingivitis in a dog gingivitis model. Gao, C., et al., Effects of Cimetidine on Progression of Naturally Occurring Periodontitis in Beagle Dogs (2002).¹ These studies demonstrated that cimetidine significantly reduced gingival inflammation and gingival bleeding with no antiplaque activity or increase in tooth stain. Snider, A. G., Evaluation of H2-Receptor Antagonists Cimetidine, Ranitidine and Famotidine in an In Vivo Gingivitis Model (2002).² Abstract available at http://iadr.confex.com/iadr/2005SanDiego/techprogram/abstract_{—}11483.htmAbstract available at http://iadr.confex.com/iadr/2005SanDiego/techprogram/abstract_{—}11258.htm

In a dose response study in the same dog gingivitis model, it was shown that cimetidine HCL had significant anti-gingivitis activity that increased with increasing dose. However, in a prevention study in the same dog model, results were not conclusive. In a series of clinical studies in humans that evaluated a topical cimetidine rinse on neutrophil function in the gingival crevice, it was shown that topical 0.5% cimetidine oral rinse enhanced antibacterial function of crevicular neutrophils with no clinical gingivitis benefit.

Selective H2 antagonists are not generally known as anti-inflammatory agents but surprisingly act to reduce inflammation. There are several problems, however, with oral topical application of H2 antagonists to the mucosal tissues of the oral cavity as described in the U.S. Pat. No. 5,294,433. First, these chemicals are not well absorbed by the gingival tissues. Second, saliva washes away the chemicals, thereby reducing their therapeutic effect.

Due to their poor absorption and the slight damage they cause to the membrane barrier, H2 antagonists can take many weeks, or even months of treatment, to have a therapeutic effect on periodontal disease. This effect has been demonstrated through extensive study of the use of H2 antagonists for the treatment of stomach ulcers and demonstrated in gingivitis models. This effect is primarily due to the modulation of the immune system over time of the H2 antagonist, not due to the immediate topical effects of the compound. H2 antagonists alone do not enhance the membrane stability and mucosa of the mouth to prevent or treat damage to the tissues. Topically-applied H2 antagonists alone have also been shown to cause irritation and cell death around permeable tissues in the mouth. H2 antagonists do not have enough antibacterial effect, which is needed in order to protect the mucosa

The buccal mucosa is a potential site for the controlled delivery of hydrophilic macromolecular therapeutic agents (biopharmaceuticals) such as peptides, oligonucleotides and polysaccharides. However, these high molecular weight drugs usually have low permeability leading to a low bioavailability, and absorption enhancers may be required to overcome this. Amir H Shojaei, Buccal Mucosa As A Route For Systemic Drug Delivery, Journal of Pharmacy and Pharmaceutical Sciences, 1998,1(1), 15-30. It has been demonstrated that encapsulation of H2 antagonist in paucillamelar liposomes, as described in U.S. Patent Publication No. 2013/0108688, could be a lower molecular weight alternative in drug delivery of H2 antagonist to the oral mucosa.

Chitosan, a cationic copolymer of glucosamine and N-acetyl-D-glucosamine, is a partially deacetylated derivative of a natural polysaccharide, chitin. Chitin is one of the most abundant carbohydrates in nature and is mostly derived from the exoskeleton of crustaceans. While chitosan is used in many applications, including pharmaceutical, its use is severely limited because it is insoluble at neutral and alkaline pH. Solubilty is only observed below pH 6.5, which is the pKa of chitosan.

Chitosan is thought to have potential as an agent for controlled release drug delivery because of its biocompatibility, biodegradability, bioactivity, and nontoxicity. However, again, a significant drawback to the use of chitosan for these purposes remains its relatively limited solubility in water. Furthermore, chitosan has limited capacity for controlled the release of an encapsulated compound and requires chemical crosslinking in order to avoid rapid dissolution of the encapsulated compounds into the gastric cavity for peroral formulations. Other investigators have made cellulose/chitosan microspheres in order to solve some of these problems. These microspheres were shown to adhere to the gastric mucous layer and have potential for controlled drug release, however the cellulose comprised the outer layer of the microspheres. Zhou, H., et al, In Vivo and In Vitro Evaluation of Mucoadhensiveness [sic] of Chitosan/Cellulose Acetate Microspheres, Journal of Biomedical Materials Research, 1146-1153 (2007).

Chitosan has been used topically to treat periodontal disease. Chitosan rinses have been shown to be effective in reducing plaque formation and counts of salivary mutans streptococci after a 14-day rinsing period. Mutans streptococci is a group of oral streptococci that is closely related to Streptococcus mutans. Streptococcus mutans is a facultatively anaerobic, Gram positive coccus-shaped bacterium that is commonly found in the human oral cavity and is a significant contributor to tooth decay. Despite the fact that chitosan showed initial promise as an effective anti-plaque agent for use in oral hygiene products, there are a number of issues with delivering the chitosan to the oral tissues in an effective manner. Chitosan washes away easily from oral tissues, and thus reduces efficacy and cannot act to reduce oral inflammation. Furthermore, chitosan is poorly soluble in water. Low molecular weight forms of chitosan with a molecular mass of 5-6 kDa and a degree of deacetylation of between about 50% and 60% are more effective than high molecular weight chitosans. Sanom, H., et al., Effect of Molecular Mass and degree of Deacetylation of chitosan on adsorption of Streptococcus sobrinus 6715 to saliva treated hydroxyapatite, Bull, Tokyo Dent. Coll, 43(2): 75-82 (2002). Chitosan rinses have also been shown to increase the permeability of the oral mucosa reducing the membrane stability and resistance to recovery.

In light of the difficulties in delivering H2 antagonists and chitosan in a manner that results in therapeutic effect, there is a need in the art for compositions that are capable of doing so. Previous attempts at remedying this problem have focused on liposomal delivery of these active agents, as described in U.S. Patent Publication No. US 2011/0135716 A1. However, further investigation showed that these prior art liposomal formulations were unsuitable for commercial use as an antibacterial and anti-inflammatory in the treatment of oral inflammatory disesases. These liposomal formulations had very poor shelf stability, required special storage conditions, and could not be mixed with the standard other components typical of topical oral products, such as flavorings, preservatives, antibacterials (such as low molecular weight chitosan) and other active ingredients of interest.

As a result, there is a need in the art for stable formulations that are suitable for topical oral administration and that enhance the delivery, effectiveness and life cycle of a drug, preferably an H2 antagonist.

### BRIEF DESCRIPTION OF THE INVENTION

Some embodiments of the present invention relate to novel polycationic electrically charged compositions that are capable of forming a localized oral membrane barrier to prevent and treat disease progression in oral infections. In some aspects, the compositions comprise a mucosal adhesive and a positive charge ingredient with a stabilizer having a mechanical action at the membrane barrier of the periodontal tissues.

Other embodiments includes novel methods for the delivery of a polycationic mucosal-adhesive polymer such as low molecular weight chitosan in combination with an H2 antagonist in a localized delivery system. Still other embodiments include co-administration of other ingredients along with said composition. The present invention also relates to method of administration of a composition for the treatment of skin and oral infectious diseases.

The compositions of the invention are capable of protecting and maintaining the barrier function of the oral gingival tissues against the onslaught of plaque pathogens. The clinical consequence of maintaining this barrier protection is preventing existing disease from further invasion and maintaining the integrity of periodontal tissues. This innovation enhances periodontal health and/or increases the protective capacity of the periodontium.

The compositions may be used as a stand-alone therapy, such as solutions, mouth-washes, dispersion, suspensions, emulsions, mixtures, lotions, liniments, gels, jellies, ointments, creams, pastes including toothpastes, dentifrices, gels, hydrogels, aerosols, sprays including mouth sprays, powders including tooth powders, granules, granulates, lozenges, salve, chewing gum, pastilles, sachets, mouthwashes, tablets, including effervescent tablets, dental floss, composites, mesh, plasters, bandages, sheets, foams, films, sponges, dressings, drenches, bioabsorbable patches, sticks, and the like, that need not be strictly tied to mechanical debridement to be effective.

### DETAILED DESCRIPTION OF THE INVENTION

The present invention provides for a mucoadhesive multilayer liposome comprising (a) a liposomal particle; (b) the liposomal particle being coated with a mucoadhesive polymer such as low molecular weight chitosan and (c) an H2 antagonist encapsulated as a stabilizer with optionally one or more pharmaceutically acceptable carriers and/or excipients. The present invention overcomes the problems seen in the prior art with administration of H2 antagonists to oral and other tissues, especially the large particle size of the prior art compositions with liposomes and chitosan encapsulated. The use of low molecular weight chitosan to coat the mucoadhesive multilayer liposome compositions of the instant invention results in a mucoadhesive liposome particle small enough to effectively deliver drug to target tissues for a sustained period of time.

The disclosed compositions preferably comprise a host modulator and a mucoadhesive antibacterial agent. In some embodiments the host modulator is an H2 antagonist. Non-limiting examples of suitable H2 antagonists are cimetidine, ranitidine, famotidine, and nizatidine. The concentration of the H2 antagonist ranges between about 0.01% and about 10%, preferably between about 0.1% w/w and about 5% w/w. A number of classes of compounds have been evaluated as host modulation agents. In some embodiments, the host modulator is selected from the classes of drugs including nonsteroidal antiinflammatory drugs (NSAIDs), bisphosphonates, tetracyclines, cytokine antagonists, nitric oxide synthase inhibitors, enamel matrix tetracyclines, growth factors and bone morphogenetic proteins.

The composition of the present invention may contain additionally an inhibitor of cyclo-oxygenase (COX)-1 and/or COX-2 enzymes.

The disclosed novel compositions are also suitable for formulating for standard oral applications because they can be combined with excipients including, but not limited to, flavorings, preservatives, and other active ingredients, including, but not limited to, nutrients, vitamins, omega-3 fatty acids, hyaluronic acid, disinfectants of the oral cavity, steroidal or non-steroidal anti-inflammatories, wound healing agents, analgesics, antimicrobials, and antihistamines.

The composition of the invention may further comprise the usual cosmetic or dermatological adjuvants and/or additives such as preservatives/antioxidants, fatty substances/oils, water, organic solvents, silicones, thickeners, softeners, emulsifiers, additional, antifoaming agents, moisturizers, fragrances, sweeteners, surfactants, fillers, sequestering agents, anionic, cationic, nonionic or amphoteric polymers or mixtures thereof, propellants, acidifying or basifying agents, dyes, colorants, stabilizers, whitening agents, antibacterial agents, preservatives active ingredients or any other ingredients usually formulated into cosmetics or drugs. The additives and/or additional active ingredients can, based on the desired product, easily be chosen by a person skilled in the art.

Physiologically acceptable carriers or excipients for use with the inventive pharmaceutical compositions can be routinely selected for a particular use by those skilled in the art. These include, but are not limited to, solvents, buffering agents, inert diluents or fillers, suspending agents, dispersing or wetting agents, preservatives, stabilizers, chelating agents, emulsifying agents, anti-foaming agents, gel-forming agents, ointment bases, penetration enhancers, humectants, emollients, and skin protecting agents.

Examples of solvents are water, alcohols, vegetable, marine and mineral oils, polyethylene glycols, propylene glycols, glycerol, and liquid polyalkylsiloxanes. Inert diluents or fillers may be sucrose, sorbitol, sugar, mannitol, microcrystalline cellulose, starches, calcium carbonate, sodium chloride, lactose, calcium phosphate, calcium sulfate, or sodium phosphate. Examples of buffering agents include citric acid, acetic acid, lactic acid, hydrogenophosphoric acid, and diethylamine. Suitable suspending agents are, for example, naturally occurring gums (e.g., acacia, arabic, xanthan, and tragacanth gum), celluloses (e.g., carboxymethyl-, hydroxyethyl-, hydroxypropyl-, and hydroxypropylmethyl-cellulose), alginates and chitosans. Examples of dispersing or wetting agents are naturally occurring phosphatides (e.g., lecithin or soybean lecithin), condensation products of ethylene oxide with fatty acids or with long chain aliphatic alcohols (e.g., polyoxyethylene stearate, polyoxyethylene sorbitol monooleate, and polyoxyethylene sorbitan monooleate).

Preservatives may be added to a pharmaceutical composition of the invention to prevent microbial contamination that can affect the stability of the formulation and cause infection in the patient. Suitable examples of preservatives include parabens (such as methyl, ethyl, propyl, p-hydroxybenzoate, butyl, isobutyl, and isopropylparaben), potassium sorbate, sorbic acid, benzoic acid, methyl benzoate, phenoxyethanol, bronopol, bronidox, MDM hydantoin, iodopropynyl butylcarbamate, benzalconium chloride, cetrimide, and benzylalcohol. Examples of chelating agents include sodium EDTA and citric acid.

Examples of emulsifying agents are naturally occurring gums, naturally occurring phosphatides (e.g., soybean lecithin; sorbitan mono-oleate derivatives), sorbitan esters, monoglycerides, fatty alcohols, and fatty acid esters (e.g., triglycerides of fatty acids). Anti-foaming agents usually facilitate manufacture, they dissipate foam by destabilizing the air-liquid interface and allow liquid to drain away from air pockets. Examples of anti-foaming agents include simethicone, dimethicone, ethanol, and ether.

Examples of gel bases or viscosity-increasing agents are liquid paraffin, polyethylene, fatty oils, colloidal silica or aluminum, glycerol, propylene glycol, carboxyvinyl polymers, magnesium-aluminum silicates, hydrophilic polymers (such as, for example, starch or cellulose derivatives), water-swellable hydrocolloids, carrageenans, hyaluronates, and alginates. Ointment bases suitable for use in the compositions of the present invention may be hydrophobic or hydrophilic, and include paraffin, lanolin, liquid polyalkylsiloxanes, cetanol, cetyl palmitate, vegetable oils, sorbitan esters of fatty acids, polyethylene glycols, and condensation products between sorbitan esters of fatty acids, ethylene oxide (e.g., polyoxyethylene sorbitan monooleate), and polysorbates.

Examples of humectants are ethanol, isopropanol glycerin, propylene glycol, sorbitol, lactic acid, and urea. Suitable emollients include cholesterol and glycerol. Examples of skin protectants include vitamin E, allatoin, glycerin, zinc oxide, vitamins, and sunscreen agents.

The pharmaceutical compositions of the invention may, alternatively or additionally, comprise other types of excipients including, thickening agents, bioadhesive polymers, and permeation enhancing agents.

Thickening agents are generally used to increase viscosity and improve bioadhesive properties of pharmaceutical compositions. Examples of thickening agents include, but are not limited to, celluloses, polyethylene glycol, polyethylene oxide, naturally occurring gums, gelatin, karaya, pectin, alginic acid, and povidone. Particularly interesting are thickening agents with thixotropic properties (i.e., agents whose viscosity is decreased by shaking or stirring). The presence of such an agent in a pharmaceutical composition allows the viscosity of the composition to be reduced at the time of administration to facilitate its application to the site of interest (e.g., to the gingiva or periodontal pocket) and, to increase after application so that the composition remains at the site of administration.

In embodiments where an inventive pharmaceutical composition is intended to be applied on skin, bioadhesive polymers are useful to hydrate the skin and enhance its permeability. Bioadhesive polymers can also function as thickening agents. Examples of bioadhesive polymers include, but are not limited to, pectin, alginic acid, chitosan, polysorbates, polyethylene glycol), oligosaccharides and polysaccharides, cellulose esters and cellulose ethers, and modified cellulose polymers. Permeation enhancing agents are vehicles containing specific agents that affect the delivery of active components through the skin. Permeation enhancing agents include solvents, such as alcohols (e.g., ethyl alcohol, isopropyl alcohol), dimethyl formamide, dimethyl sulfoxide, 1-dodecylazocyloheptan-2-one, N-decylmethylsulfoxide, lactic acid, N,N-diethyl-m-toluamide, N-methylpyrrolidone, nonane, oleic acid, petrolatum, polyethylene glycol, propylene glycol, salicylic acid, urea, terpenes, and trichloroethanol) and surface active compounds.

In embodiments where an inventive pharmaceutical composition is intended to be applied on skin, the pharmaceutical composition may be packaged as kits comprising a container including the composition, optionally admixed with physiologically acceptable carriers or excipients, and at least one dressing, wherein the dressing is to be applied to cover the skin site following local administration of the content of the container to the site. The term “dressing” refers to any covering designed to protect a skin site. The term includes porous and non-porous coverings, woven and non-woven coverings, absorbent coverings, and occlusive coverings. The dressing may also be used as a delivery system for the pharmaceutical composition of the invention. For example, the pharmaceutical composition may be incorporated into or coated onto the dressing (e.g., by dipping the dressing in or spraying the dressing with the pharmaceutical composition of the invention).

In embodiments where an inventive pharmaceutical composition is intended to be administered to the oral cavity, the composition may desirably comprise other components, such as, for example, topical oral carriers. Such carriers include, but are not limited to, anticaries agents, antiplaque agents, anticalculus agents, anti-inflammatory agents, dental abrasives, flavoring agents, sweetening agents, binders, humectants, thickening agents, buffering agents, preservatives, coloring agents, and pigments, flavorants, fillers, stabilizers, ethanol and water.

In view of the instant disclosure, the compositions of the invention can be prepared according to known methods, described for example in Remington, The Science and Practice of Pharmacy, 20th Edition, typically in forms useful for the treatment of afflictions of the oral mucous membranes (periodontitis and the like).

Unlike the prior art liposomal delivery systems, the compositions disclosed herein may be formulated into a variety of products. Depending on the mode of administration, the inventive pharmaceutical compositions may be in the form of liquid, solid, or semi-solid dosage preparation. Examples include, but are not limited to, solutions, mouth-washes, dispersion, suspensions, emulsions, mixtures, lotions, liniments, gels, jellies, ointments, creams, pastes including toothpastes, dentifrices, gels, hydrogels, aerosols, sprays including mouth sprays, powders including tooth powders, granules, granulates, lozenges, salve, chewing gum, pastilles, sachets, mouthwashes, tablets, including effervescent tablets, dental floss, composites, mesh, plasters, bandages, sheets, foams, films, sponges, dressings, drenches, bioabsorbable patches, sticks, and the like. The compositions are effective in protecting the oral tissues while ensuring effective, therapeutic delivery of the active ingredients.

The coating of the liposome with a mucoadhesive polymer such as low molecular weight chitosan allows for Cimetidine or the other antagonists H2 to remain for a longer time on mucous membranes, in particular on the oral mucous membranes, thus enhancing the penetration of the medicament with evident, advantageous therapeutic benefits. The concentration of the low molecular weight chitosan ranges between about 0.001% and about 10%, and is preferably between about 0.01% w/w and about 5% w/w. The mucoadhesive action of the compositions is promoted and further increased by the presence of water; water based mouth-washes are therefore particularly preferred. The physiological water present in the oral cavity also ensures an increase in mucoadhesivity even to compositions with very low—if any—water content. Another advantage of these compositions is that they may be alcohol free. Presently, most of the compositions available in liquid form to treat periodontal disease require the inclusion of at least some alcohol for its antiseptic and antibacterial properties.

The compositions of the invention can further comprise other active principles useful for the topical treatment of oral mucous membranes, described for example in Martindale, The Complete Drug Reference, 34^{th} Edition. Examples of said further active principles comprise disinfectants of the oral cavity such as Propolis, chlorhexidine, benzalkonium, cetylpyridinium, Triclosan, silver and derivatives; steroidal or non steroidal anti-inflammatories such as Cortisone emisuccinate, Diclofenac, Ibuprofen, Ketoprofen; wound-healing agents such as Aloe vera, allantoin and derivatives, Liquorice and derivatives; analgesics such as Lidocaine and Benzydamine; antimicrobials, antihistaminics, hyalauronic acids, and omega-3 fatty acids.

Also claimed herein are methods of administering the disclosed compositions to treat or prevent periodontal pathologies in mammals, preferably humans. Also contemplated are veterinary uses for the compositions, particularly administration to dogs, pets, and farm animals. The compositions of the present invention are effective and provide continuous protection when administered to a patient between 1 and 4 times a day.

The term “topical composition” as used herein refers to any cosmetic or pharmaceutical composition that can be topically applied to mammalian keratinous tissue.

Embodiments of the present invention include a composition comprising a mucoadhesive multilayer liposome (MML) comprising (a) a liposomal particle; (b) a mucoadhesive polymer; and (c) an H2 antagonist encapsulated as a stabilizer. In some embodiments, the mucoadhesive polymer is coated on the liposome. In still others mucoadhesive the polymer is low molecular weight chitosan.

In some embodiments the liposomal particle is a paucillamelar liposome or multilayer liposome. In others the composition of claim 1 wherein the liposomal particle is comprised of ethoxydiglycol, glyceryl dilaurate, propylene glycol dicaprylate/dicaprate, cholesterol, cetearyl alcohol and cetearyl glucoside.

In some compositions of the invention, the H2 receptor antagonist constitutes between 0.01 and 10% of the composition. In yet other embodiments, the H2 antagonist is cimetidine. In still others, the compositions further comprise one or more active ingredients selected from the group consisting of disinfectants of the oral cavity, steroidal or non-steroidal anti-inflammatories, wound healing agents, analgesics, antimicrobials, host mediators, antihistamines, hyalauronic acids, or omega-3 fatty acids.

In still further embodiments, the composition forms a protective film over one or more of the oral mucosa, skin, keratinized tissue, and epithelial basal cells. In other embodiments, the composition is suitable for topical oral administration. In some aspects of the invention, the composition is topically administered between 1 and 4 times per day.

These compositions may be formulated into a product selected from the group consisting of solutions, mouth-washes, dispersion, suspensions, emulsions, mixtures, lotions, liniments, gels, jellies, ointments, creams, pastes including toothpastes, dentifrices, gels, hydrogels, aerosols, sprays, mouth sprays, powders, tooth powders, granules, granulates, lozenges, salve, chewing gum, pastilles, sachets, mouthwashes, tablets, effervescent tablets, dental floss, composites, mesh, plasters, bandages, sheets, foams, films, sponges, dressings, drenches, bioabsorbable patches, sticks.

Some aspects of the invention include methods of preventing gingival or periodontal pathology characterized by inflammation and pain comprising administering an effective amount of the composition. Others include methods of treating an existing gingival or periodontal pathology characterized by inflammation and pain comprising administering a safe and/or effective amount of the composition of claim 1. In some embodiments, the pathology is gingivitis. In others, the disease is periodontitis around the teeth and/or dental implants.

Other diseases and conditions may be treated with the compositions of the present invention. In some embodiments, the condition is selected from the group consisting of psoriasis, atopic eczema, urticaria, allergic reaction, warts, burn itch, squamos cell carcinoma, skin diseases or orthopedic diseases.

### Multilayer Liposome

In a preferred embodiment the liposome of the invention are multilayer liposomes. More preferably they are non-phospholipid paucilamellar vesicles. Such paucilamellar liposomes (PLs) consist of between two to seven lipid bilayers that surround an unstructured space occupied by a large amorphous core of hydrophilic or hydrophobic materials.

The molecules that are used to form such PLs generally have a hydrophilic head group attached to a hydrophobic tail and include long-chain fatty alcohols and derivatives, long chain acids, long chain amino acids and glycerolipids. The bilayered membranes may be formed of many biocompatible, single tailed amphiphiles as well as individually chosen phospholipids. The bi-layers have the fatty acid tails pointing into the membranes' interiors and the polar head groups pointing outward. The polar groups present at one surface of the membrane point towards the interior of the vesicle while those at the other surface point toward their external environment.

Any water-soluble molecules that have been mixed with the water at the time of manufacturing of the vesicle, are incorporated into the aqueous spaces between the multiple layers of the lipid bilayer membrane. Similarly, any lipid soluble molecules added at the time of vesicle formation are incorporated inside the vesicle core. The amorphous core accounts for most of the volume of the vesicle, incorporating water soluble, water immiscible and small solid particles. These microvesicles are stable over a wide pH range of 2-13 and temperature range of liquid nitrogen to above the boiling point of water.

Many researchers have reported on the problems with oral administration of peptide drugs—i.e. the physical and chemical instability of peptide drugs in the gastrointestinal (GI) tract or biological environment, the absorption at the site of administration, the degradation by enzymes and so on (Takeuchi et al. 1996, 2003; Kotze et al. 1997; Iwanaga et al. 1997, 1998). Many strategies have been adopted to remedy these problems, such as the use of mucoadhesive polymers in tablet dosage form(Bernkop-Schnurch et al. 1998), the synthesis and modification of mucoadhesive polymers to improve drug efficiency (vander Merwe et al. 2004), the development of special dosage forms by utilizing micro- or nano-particles or nanocapsules as a drug carrier (Aboubakar et al.1999; Sakuma et al. 2002; Agnihotri et al. 2004;

Hu et al. 2004), the use of pH-sensitive hydrogel nanospheres and copolymer network as carriers to the targeting site (Torres-Lugo and Peppas 2002; Ichikawa and Peppas 2003) and the use of liposomal systems and surface-coated liposomal systems as drug delivery devices (Takeuchi et al. 1996, 2001, 2003, 2005; Kim et al. 1999; Katayama et al. 2003). Mucoadhesive polymers have been widely used to improve the delivery of orally administered protein and peptide products. However, the prior art formulations have not been successful in treating oral infection due the problems described in the prior art. Disclosed herein for the first time are oral compositions for topical application wherein the composition comprises a multilayer liposome comprising an H2 antagonist, wherein the multilayer liposome is coated with low molecular chitosan.

Consequently, to overcome the problems in treating periodontal disease, we have developed mucoadhesive multilayer liposomes by coating liposomal particles with a mucoadhesive polymer, such as low molecular weight chitosan and an H2 antagonist encapsulated as a stabilizer. Specifically, we have shown that a topical H2 antagonist and low molecular weight chitosan in a paucilamellar liposome formulation unexpectedly prevents periodontal inflammation progression.

### EXAMPLES

Novel polymer structures were prepared using the following general components (a) A base liposome; a coating comprising a polymer—initially low molecular weight chitosan; and (c) cimetidine—were prepared with the following structures. Initial experiments were done using the following:

| Component of | |
|---|---|
| mucoadhesive | |
| multilayer liposome | Reagent |
| Coating | Low-molecular weight chitosan (LCS) |
| Liposome | Ethoxydiglycol |
| | Glyceryl Dilaurate |
| | Propylene Glycol Dicaprylate/Dicaprate |
| | Cholesterol |
| | Cetearyl Alcohol & Cetearyl Glucoside |
| H2 Antagonist | Cimetidine |

### Preparation of Low Molecular Weight Chitosan-Coated Liposomes (LCS-Lips)

Cimetidine liposomes (cimetosomes) were prepared by first dissolving cimetidine in deionized (“DI”) water at 1.65 mg/ml in order to generate a stock solution of cimetidine. Next, Ethoxydiglycol, Glyceryl Dilaurate, Propylene Glycol Dicaprylate/Dicaprate, Cholesterol, and Cetearyl Alcohol & Cetearyl Glucoside were all weighed into an Erlenmeyer flask. The components of the flask were stirred with a magnetic stirrer while heated between 20-40° C. for 1-4 hours until dissolved. Once dissolved, the cimetidine solution was weighed into the Erlenmeyer flask containing the Ethoxydiglycol, Glyceryl Dilaurate, Propylene Glycol Dicaprylate/Dicaprate, Cholesterol, and Cetearyl Alcohol & Cetearyl Glucoside. This mixture was stirred with the magnetic stirrer at ambient temperature for approximately ten minutes.

The solution was then processed by micro-emulsification. Five passed through the micro-emulsifier were performed on the product to make the cimetisomes. The resulting material was a stable emulsion of cimetisomes.

A 0.04% stock solution of low molecular weight was prepared by weighing low molecular weight chitosan into an Erlenmeyer flask and diluting with DI water at a ratio of 40 mg chitosan per 100 ml DI water. The chitosan is not water soluble at neutral pH, so HCl was titrated into the stock solution until approximately pH 3. After allowing the chitosan to stir for approximately one hour at pH 3, the chitosan dissolved to give a clear stock solution. A 0.004% low molecular weight chitosan stock solution was then prepared by doing a 10 time dilution of the 0.04% chitosan solution into DI water.

A portion of the 0.004% chitosan solution was mixed with an equal volume of cimetisomes to give a final chitosan concentration of 0.002%. The two phases were vortexed for approximately 20 seconds to mix the materials. 0.002% chitosan was targeted as the lowest usable concentration, and the solution must demonstrate a stable emulsion with no phase separation for at least 48 hours, which was in fact the case. The emulsion was stable for a time greater than 48 hours.

To determine the highest stable concentration of chitosan, different solutions at varying concentrations of chitosan were prepared. Concentrations of medium molecular weight chitosan were prepared at 0.004%, 0.008%, 0.01%, 0.013%, 0.02%, and 0.04%. Concentrations of low molecular weight chitosan were prepared at 0.01%, 0.013%, 0.016%, 0.02%, and 0.04%. All of these concentrations were prepared from a dilution of 0.04% stock solutions of chitosan. Each diluted concentration was mixed with an equal volume of cimetisomes as mixed above. Following the same acceptance criteria, the highest stable chitosan concentration was observed to be 0.005% chitosan for low MW chitosan and for medium MW chitosan. Higher concentrations would cause the emulsion to phase separate.

Once the highest concentration had been determined, a 0.01% chitosan solution was prepared at pH 3. This solution was divided and one portion was adjusted to approximately pH 5 with NaOH. The pH could not be adjusted higher because the chitosan began to come out of solution. Both portions of the chitosan solution were mixed with an equal volume of cimetosomes and vortexed. This portion of the experiment is being done to determine if pH has an effect on causing phase separation of the product. Phase separation was not observed in either sample, and separation did not appear to be caused by pH variations. Both solutions had a final pH of 6-7.

Particle size was checked and compared to the cimetisomes' particle size. A detailed comparison of the data is shown in TABLE A.

Particle size:
Cimetisomes 248.3 nm effective diameter
Chitosan coated cimetisomes 335.7 nm effective diameter
Chitosan coated cimetisomes
(with pH adjusted chitosan) 249.6 nm effective diameter

TABLE A

| | | | Effective | |
|---|---|---|---|---|
| | Preparation | Particle Size | Particle | Poly- |
| | Date | test date | size (nm) | dispersity |
| Cimetisomes | Oct. 21, 2011 | Oct. 21, 2011 | 240.2 | 0.106 |
| Cimetisomes | Oct. 21, 2011 | Oct. 28, 2011 | 246.5 | 0.136 |
| Cimetisomes | Oct. 21, 2012 | May 24, 2012 | 252 | 0.125 |
| Cimetisomes | Oct. 21, 2011 | Oct. 28, 2011 | 249.9 | 0.13 |
| (Stored at 40° C.) | | | | |
| Cimetisomes | Oct. 21, 2011 | Dec. 13, 2011 | 311.1 | — |
| (Stored at 40° C.) | | | | |
| Cimetisomes | May 7, 2012 | May 7, 2012 | 248.1 | 0.145 |
| Cimetisomes | May 16, 2012 | May 16, 2012 | 248.3 | 0.078 |
| Cimetisomes, 0.005% | May 17, 2012 | May 17, 2012 | 327.4 | 0.274 |
| Med MW Chitosan | | | | |
| Cimetisomes, 0.005% | May 17, 2012 | May 21, 2012 | 323.1 | 0.261 |
| Med MW Chitosan | | | | |
| Cimetisomes, 0.005% | May 17, 2012 | May 17, 2012 | 250.4 | 0.08 |
| Med MW Chitosan, | | | | |
| adjusted Chitosan to | | | | |
| pH5 prior to mixing | | | | |
| Cimetisomes, 0.008% | May 17, 2012 | May 17, 2012 | 528.9 | 0.336 |
| Low MW Chitosan | | | | |
| Cimetisomes, 0.008% | May 17, 2012 | May 17, 2012 | 249.6 | 0.094 |
| Low MW Chitosan, | | | | |
| adjusted Chitosan to | | | | |
| pH5 prior to mixing | | | | |
| Cimetisomes, 0.065% | May 17, 2012 | May 17, 2012 | 335.7 | 0.275 |
| Low MW Chitosan | | | | |
| Cimetisomes, 0.005% | May 17, 2012 | May 21, 2012 | 276.8 | 0.184 |
| Low MW Chitosan | | | | |

### Stability at 48 Hours With Different Weight Chitosan

The low molecular weight chitosan and the medium molecular weight chitosan behaved the same. The highest stable concentration of both was 0.005% chitosan mixed with cimetisomes. Both molecular weights of chitosan coated cimetisomes were observed to be stable after 48 hours.

### Paucillamelar Liposomes

In a preferred embodiment of the liposomes of the invention are paucillamelar leptosomes (PLs).

PLs are found to have several beneficial characteristics over other types of liposomes. They are generally composed of a multi bilayered vesicle with a high capacity central core. The surface of the liposome can be negative, positive or neutrally charged. They provide for a continuous release of active ingredient and the inner amorphous core can be loaded up to 80-85% with active ingredient and can be produced with specific size ranges.

Additionally, PLs have the ability to adhere to the skin or hair from the bilayers through the aqueous suspension shaft depending on varying conditions of the vesicle separating the bilayers

These characteristics offer several advantages in the delivery of the active ingredient present in the core including (a) storage stability and the stability of the active ingredient; (b) capability to deliver both hydrophilic as well as hydrophobic products in the same formulation.

Paucillamelar liposomes (PLs) can be prepared from a mixture of monoester of polyoxyethylene fatty acids, cholesterol and free fatty acids at 74/22/4 ratio. The PLs consisted of two to seven bi-showing inner layered shells that surround an unstructured space occupied by a large amorphous core of hydrophilic molecules (or alternatively any water soluble molecules) that have been mixed with the hydrophobic materials and/or water at the time of manufacturing of the vesicle.

The paucilamellar lipid vesicles formed rapidly in less than 1 second and can be removed from the chamber through the discharge orifice axially located to the chamber. These lipid vesicles can include non-phospholipid surfactants, charge producing agent and a targeting molecule. Non-phospholipid paucilamellar lipid vesicles made of non-phospholipid surfactant material (e.g., polyoxyethylene cetyl ether, polyoxyethylene lauryl ether, glyceryl monostearate and poly oxyethylene glyceryl stearate) and containing an antioxidant, involve formation of a lipophilic phase containing a mixture of several lipophilic components. Various studies that non-phospholipid vesicles proved to have more encapsulation efficiency and shows better targeting and sustained release of active ingredients.

The PLs have uniformity of size. Their encapsulation efficiency varies from 100% for groups present at one surface of the membrane point lipid moieties to 85% for aqueous materials.

1. A composition comprising a mucoadhesive multilayer liposome (MML) comprising (a) a liposomal particle; (b) a mucoadhesive polymer; and (c) an H2 antagonist encapsulated as a stabilizer.

2. The composition of claim 1 wherein the mucoadhesive polymer is coated on the liposome.

3. The composition of claim 2 wherein the polymer is low molecular weight chitosan.

4. The composition of claim 1 wherein the liposomal particle is a paucillamelar liposome or multilayer liposome.

5. The composition of claim 1 wherein the liposomal particle is comprised of ethoxydiglycol, glyceryl dilaurate, propylene glycol dicaprylate/dicaprate, cholesterol, cetearyl alcohol and cetearyl glucoside

6. The composition of claim 1 wherein the H2 antagonist is cimetidine.

7. The composition of claim 3 wherein the composition is suitable for topical oral administration.

8. The composition of claim 3 wherein the composition is formulated into a product selected from the group consisting of liquid forms, gels, toothpastes, patches, solid forms.

9. A method of preventing gingival or periodontal pathology characterized by inflammation comprising administering an effective amount of the composition of claim 1.

10. A method of preventing gingival or periodontal pathology characterized by inflammation comprising administering an effective amount of the composition of claim 3.

11. A method of treating an existing gingival or periodontal pathology characterized by inflammation comprising administering an effective amount of the composition of claim 1.
